(19) United States
(12) Patent Application Publication
(10) Pub. No.: US 2025/0218222 A1
(43) Pub. Date: Jul. 3, 2025

(54) SYSTEMS AND METHODS FOR AUTOMATIC HAND GESTURE RECOGNITION

(71) Applicant: Shanghai United Imaging Intelligence Co, Ltd., Shanghai (CN)

(72) Inventors: Zhongpai Gao, Rowley, MA (US); Abhishek Sharma, Boston, MA (US); Meng Zheng, Cambridge, MA (US); Benjamin Planche, Briarwood, NY (US); Ziyan Wu, Lexington, MA (US); Terrence Chen, Lexington, MA (US); Fan Yang, Shanghai (CN); Yuchun Liu, Shanghai (CN)

(73) Assignee: Shanghai United Imaging Intelligence Co, Ltd., Shanghai (CN)

(21) Appl. No.: 18/398,593

(22) Filed: Dec. 28, 2023

(51) Int. Cl. G06V 40/20; G06F 3/01

(52) U.S. Cl. CPC G06V 40/28; G06F 3/017

(57) ABSTRACT

An apparatus in accordance with embodiments of the present disclosure may obtain an image depicting one or more hands of a person in a medical environment; and detect, using a first machine learning (ML) model, a plurality of 2D landmarks associated with a hand of the person depicted in the image. The apparatus may further determine, using a second ML model, 3D features of the hand of the person based on the plurality of 2D landmarks. The apparatus may determine a gesture indicated by the hand of the person based on the 3D features of the hand predicted using the second ML model. Alternatively, in determining the 3D features of the hand, the system may stack the plurality of 2D landmarks across a sequence of image frames in a video, and use a third ML model to determine the 3D features of the hand based on the stacked 2D landmarks.

### BACKGROUND

Hand gesture recognition plays an important role in human-machine interaction. Taking a medical environment as an example, a medical professional or a patient may use hand gestures to convey a variety of information including, for example, the condition of certain medical equipment (e.g., whether a scan bed is at the right height), the readiness of the patient for a scan or surgical procedure, the pain level of the patient (e.g., on a scale of 1 to 10), etc. Therefore, having the ability to automatically recognize hand gestures may allow the medical environment to operate more efficiently and with less human intervention. Conventional techniques for hand gesture recognition may be error-prone due to the complex anatomy and high dimensionality of the human hands. Accordingly, systems and methods that are capable of accurately determining the meanings of hand gestures may be desirable.

### SUMMARY

Disclosed herein are systems, methods, and instrumentalities associated with automatic hand gesture recognition. According to embodiments of the present disclosure, an apparatus may be configured to obtain an image that depicts at least a hand of a person in a medical environment and determine, based on a first machine learning (ML) model, a representation (e.g., a heatmap) of a plurality of two-dimensional (2D) landmarks of the hand as depicted in the image. The apparatus may be further configured to predict, based on a second ML model, a three-dimensional (3D) pose of the hand based at least on the representation of the plurality of 2D landmarks of the hand, and determine a gesture of the person based on the predicted 3D pose of the hand.

In examples, the apparatus may be configured to predict the 3D pose of the hand further based on the image that depicts the hand, wherein the second ML model may be configured to receive the image as a first input and the representation of the plurality of 2D landmarks of the hand as a second input. In examples, the image that depicts the hand of the person may be cropped from another image that depicts the hand in the medical environment and the image may be re-oriented based on a pre-determined direction.

In examples, the second ML model described herein may include a first portion configured to determine a first feature map associated with the image that depicts the hand of the person in the medical environment and a second feature map associated with the representation of the plurality of 2D landmarks of the hand. The second ML model may further include a second portion configured to fuse the first feature map and the second feature map, and a third portion configured to predict the 3D pose of the hand of the person based on the fused first feature map and second feature map. In examples, the second portion of the second ML model may include a self-attention module. In examples, the third portion of the second ML model may be further configured to determine a global camera translation associated with the image that depicts the hand of the person.

In examples, the apparatus described herein may be further configured to control a medical device or manipulate a medical scan image based on the determined hand gesture of the person. For example, the apparatus may recognize that the hand gesture indicates a request to zoom in or out on the medical scan image, or to rotate the medical scan image, based on which the apparatus may manipulate the medical scan image accordingly.

In examples, the image described herein may be obtained based on a video that includes a plurality of additional images depicting the hand of the person in the medical environment. In these examples, the apparatus described herein may be further configured to determine, based on the first ML model, respective plurality of 2D landmarks of the hand as depicted by each additional image of the video, and stack the respective plurality of 2D landmarks of the hand associated with each additional image of the video such that the stacked 2D landmarks reflect spatial and temporal relationships of the 2D landmarks in the video. The apparatus may then determine the 3D pose of the hand further based on the stacked 2D landmarks. In these examples, the second ML model may include a patch partitioning portion configured to divide the stacked 2D landmarks into a plurality of non-overlapping patch areas and a transformer coupled to the patch partitioning portion and configured to extract 3D features based on the plurality of non-overlapping patch areas. The 3D pose of the hand may be predicted based at least on the 3D features extracted by the transformer.

### BRIEF DESCRIPTION OF THE DRAWINGS

A more detailed understanding of the examples disclosed herein may be obtained from the following description, given by way of example in conjunction with the accompanying drawing.

FIG. 1A is a simplified diagram illustrating an example of an automatic hand gesture recognition system in accordance with one or more embodiments of the present disclosure.

FIG. 1B is a simplified block diagram illustrating example techniques that may be used to perform a hand gesture recognition task in accordance with one or more embodiments of the present disclosure.

FIG. 1C is a simplified block diagram illustrating an example machine learning model that may be used to estimate a 3D hand pose in accordance with one or more embodiments of the present disclosure.

FIG. 2A is a simplified diagram illustrating an example of 3D hand pose estimation based on a video depicting the hand in accordance with one or more embodiments of the present disclosure.

FIG. 2B is a simplified block diagram illustrating an example machine learning model that may be used to estimate 3D hand pose using videos in a hand gesture recognition task in accordance with one or more embodiments of the present disclosure.

FIG. 2C illustrates an example arrangement of 2D landmarks across multiple image frames that may be used to estimate 3D hand pose using videos in a hand gesture recognition task in accordance with one or more embodiments of the present disclosure.

FIG. 3 is a flow diagram illustrating an example procedure for automatically determining a hand gesture in accordance with one or more embodiments of the present disclosure.

FIG. 4 is a flow diagram illustrating an example procedure for estimating 3D hand pose using videos in a hand gesture recognition task in accordance with one or more embodiments of the present disclosure.

FIG. 5 is a simplified flow diagram illustrating an example process for training an artificial neural network to perform one or more of the tasks described in one or more embodiments of the present disclosure.

FIG. 6 is a simplified block diagram illustrating example components of an apparatus that may be used to perform the hand gesture recognition task described in one or more embodiments of the present disclosure.

### DETAILED DESCRIPTION

The present disclosure is illustrated by way of example, and not by way of limitation, in the figures of the accompanying drawings. A detailed description of illustrative embodiments will be described with reference to the figures. Although this description may provide detailed examples of possible implementations, it should be noted that the details are intended to be illustrative and in no way limit the scope of the application. It should also be noted that while the examples may be described in the context of a medical environment, it will be appreciated that the disclosed techniques may also be applied to other environments or use cases.

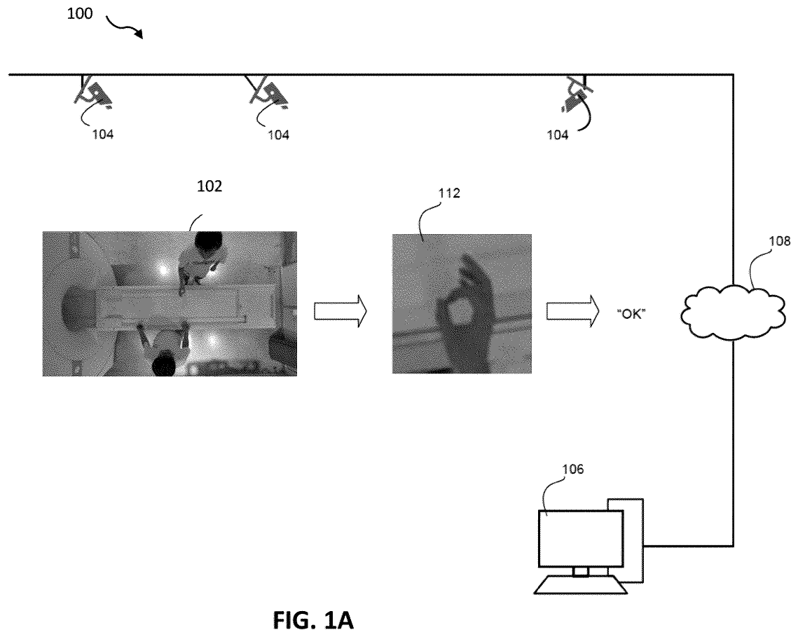

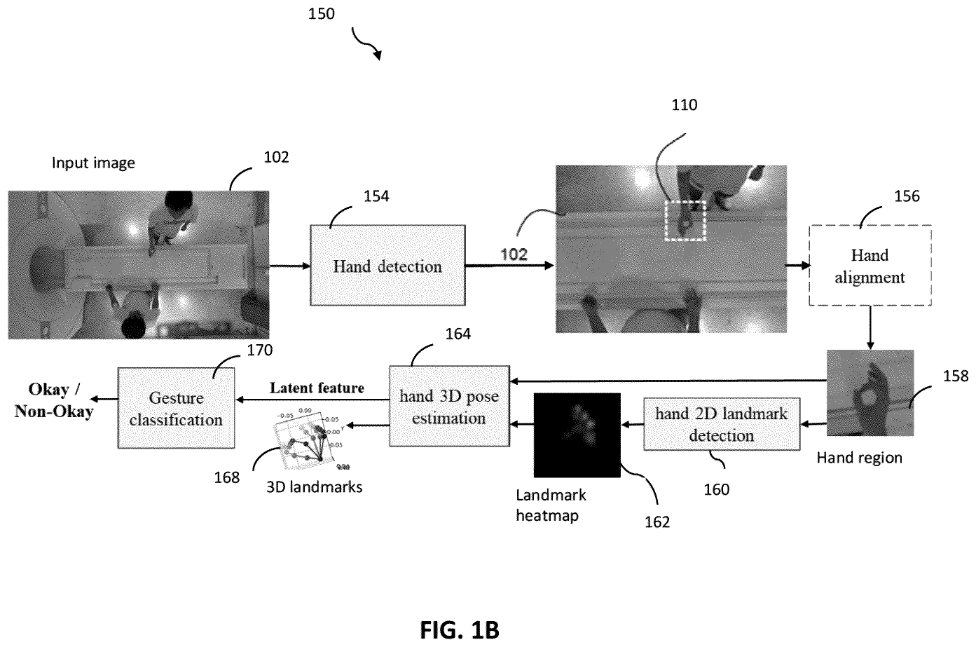

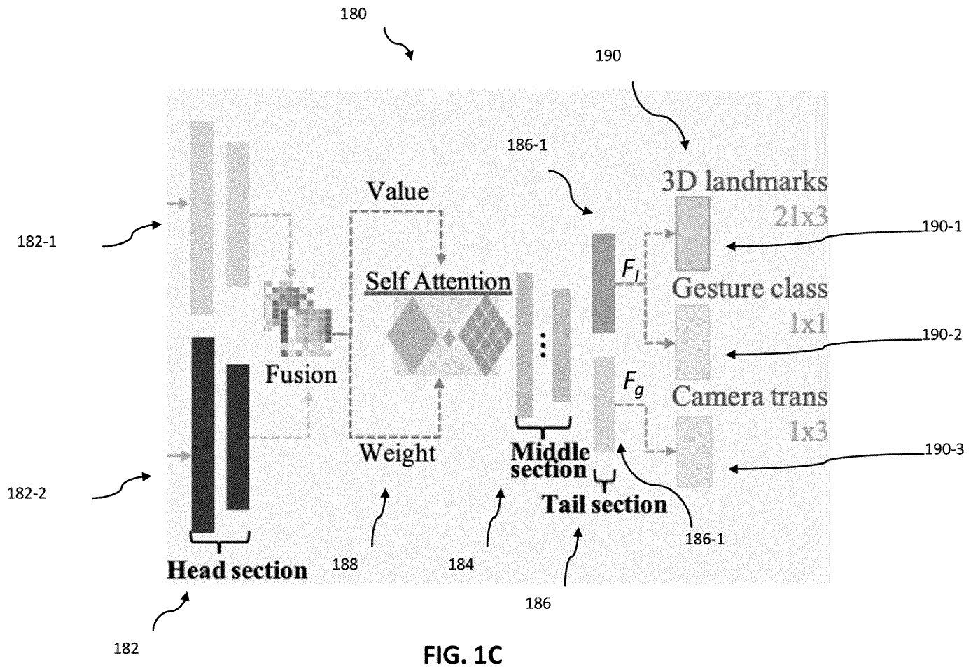

FIG. 1 illustrates an example system 100 of automatic hand gesture recognition in accordance with embodiments of the present disclosure. As shown, the hand gesture recognition may be accomplished based on an image 102 (e.g., as part of a video) of a medical environment captured by a sensing device 104 (e.g., a camera, a depth sensor, a thermal imaging sensor, a radar sensor, or a combination thereof) installed in the environment. The medical environment may be, for example, a medical environment such as a scan room (e.g., with a computed tomography (CT) or magnetic resonance imaging (MRI) scanner) or an operating room (e.g., with a surgery table). Image 102 may be a color image (e.g., a 2D color image) that depicts respective positions, shapes, and/or poses of one or more hands in the medical environment. The image may depict one or more hands of an operator of a medical device (e.g., scanning device, scanning equipment) in a session (e.g., medical scan, surgery) in the medical environment. For example, the operator may use hand gestures to cause the system to operate the medical device. The image may also depict one or more hands of a patient during a session. For example, the patient may use hand gestures to give feedback to the operator (e.g., to express pain level, readiness for a session, position of the body, such as the position where pain occurs etc.).

Sensing device 104 may be configured to provide image 102 to a processing apparatus 106 (e.g., over a wired or wireless communication link 108), while processing apparatus 106 may be configured to obtain the image 102 and determine a gesture indicated by the one or more hands based on features and/or landmarks detected in the image. For example, processing apparatus 106 may be configured to identify a hand in an area of the image (shown as a cropped image area 112 from image 102), and analyze the cropped area 112 (e.g., upon making certain adjustments to the cropped area) to determine the gesture indicated by the shape and/or pose of the hand depicted in the image. The determination may be made, for example, based on a set of pre-defined gesture classes and by matching the shape and/or pose of the hand depicted in the image to one of the pre-defined classes. For instance, upon analyzing cropped image area 112, processing apparatus 106 may determine that the shape and/or pose of the hand in the image area belong to a class of “OK” gestures, and may generate an output (e.g., a classification label) indicating the classification accordingly.

The hand gesture determination or prediction made by processing apparatus 106 may be used for a variety of purposes. For example, where processing apparatus 106 is used to detect and recognize hand gestures associated with a session in a medical environment (e.g., a scan room or an operating room), the determination made by the processing apparatus may be used to evaluate the readiness of a patient for the session (e.g., with respect to the positioning of the patient and/or other preoperative routines) or whether a medical device has been properly prepared for the session (e.g., whether a scanner has been properly calibrated and/or oriented, whether a patient bed has been set at the right height, etc.). In some examples, processing apparatus 106 may be configured to perform the aforementioned evaluation and provide an additional input indicating the output of the evaluation, while in other examples the processing apparatus may pass the hand gesture determination to another device (e.g., a device located remotely from the medical environment) so that the other device may use the determined hand gesture in an application-specific task.

System 100 as described in FIG. 1 and further herein may enable various use cases such as using hand gestures for human-computer interaction in medical environments, where the use cases may be associated with but not limited to patient positioning in a scan room, medical visualization applications, and so on. Take an MRI scanning procedure as an example, the positioning of the patient may be automated based on hand gestures. For example, once the patient is properly positioned for the procedure, a technician may post an “OK” hand gesture (e.g., using the thumb and index finger to form an “O” shape). System 100 may detect the “OK” gesture from the technician and enter a confirmation phase, where audio and visual (e.g., lights) prompts may be activated to notify the technician that the procedure is ready pending their confirmation. If the technician's intention is not to start the procedure, they may give a gesture for canceling the session.

In practice, system 100 may detect a succession of “OK” gestures before confirming the intention of the technician, making the workflow more reliable. In examples, after the confirmation, the location of the gesture center may also be determined based on the image and projected to the MRI coordinate system to indicate a target scan location. In these examples, depth value from a depth sensor and/or camera-system calibration data may be obtained during system setup (e.g., via rigid transform from the camera to the MRI system) to automatically align the center of the target scan location with the center of the MRI system, before the scan procedure is started.

It should be appreciated that the “OK” gesture used in this example is only illustrative and the example may be applicable to other suitable gestures. It should also be appreciated that system 100 may also be applied to recognize the hand gestures of a patient. Further, in variations of system 100, a feedback mechanism may be used to provide haptic, visual, or auditory cues to a user based on the recognition of the hand gestures. In some examples, an application programming interface (API) may enable system 100 and/or other applications and systems to utilize the 3D hand gesture recognition techniques described herein for medical image navigation and manipulation (e.g., to zoom in/out on a medical scan image, to rotate the medical scan image, to scale the medical scan image, etc.). In some examples, system 100 may be implemented in a medical education setting, wherein the intuitive navigation and manipulation of medical images may facilitate enhanced learning and understanding of anatomical structures. In some examples, medical image processing components in the system may support custom mappings of hand gestures to specific image manipulations based on user preferences or application requirements.

System 100 may be compatible with various medical image formats, including but not limited to MRI, CT, and X-ray images. The system may provide a more intuitive way to navigate through or manipulate medical scan images over traditional techniques that rely on the use of a keyboard or mouse, which are counterintuitive and cumbersome, especially with respect to manipulating 3D images. The systems disclosed herein may also provide increased efficiency over the traditional techniques, especially for intricate operations that are difficult to implement via computer keyboard and/or mouse. The systems disclosed herein may also provide improved precision and control over the traditional techniques, which may lead to more precise positioning, scaling, and/or rotating of medical images that is essential for accurate diagnosis and treatment planning.

The systems disclosed herein may also reduce contamination risks by reducing contact with medical devices and/or the patient. For example, using hand gestures as a contactless form of interaction may prevent multiple users from using the same mouse or keyboard, thus lowering the risk of cross-contamination. The systems disclosed herein may provide improved accessibility over traditional systems. For example, for individuals who may have difficulty using traditional input devices due to physical constraints, the systems disclosed herein offer an alternative mode of interaction that might be more accessible. The systems disclosed herein may further provide enhanced spatial perception over traditional medical image navigation systems. For example, the 3D hand gestures may enable more natural understanding and interpretation of the spatial relationships within the medical images. This may be particularly useful in educational settings, where students can manipulate images in real-time to gain a better understanding of anatomical structures. The systems disclosed herein may further provide flexibility and customization over traditional medical image navigation systems by enabling custom gestures and controls tailored to specific medical applications or user preferences, enhancing the adaptability of the system in different contexts.

It should be noted that while processing apparatus 106 may be shown in FIG. 1 as being separate from sensing device 104, those skilled in the art will appreciate that the processing apparatus may also be co-located with the sensing device (e.g., as an on-board processor of the sensing device) without affecting the functionality of the processing apparatus or the sensing device as described herein.

FIG. 1B illustrates example techniques that may be used to perform the hand gesture recognition task shown in FIG. 1A. System 150 may be implemented in system 100 (FIG. 1A) and may include a hand detection unit 154 (e.g., a software component) configured to detect one or more hands of a person (e.g., an operator or a patient) in an image 102. Hand detection unit 154 may determine a hand area including the detected hand (e.g., represented in a bounding shape such as bounding box 110). Hand detection unit 154 may additionally generate a confidence score and/or rotation angle associated with each detected hand. The rotation angle represents the angle of the detected hand relative to a pre-defined direction (e.g., the hand's upward direction). System 150 may also include hand alignment unit 156 configured to align the detected hand to upward direction (or any other pre-defined direction).

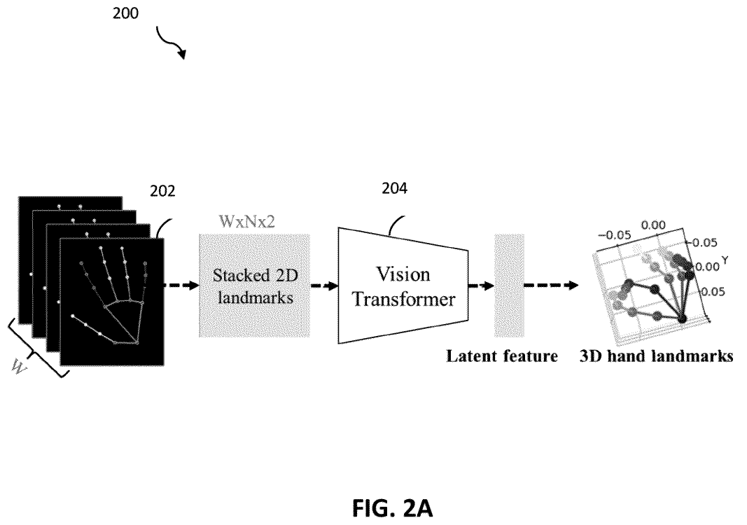

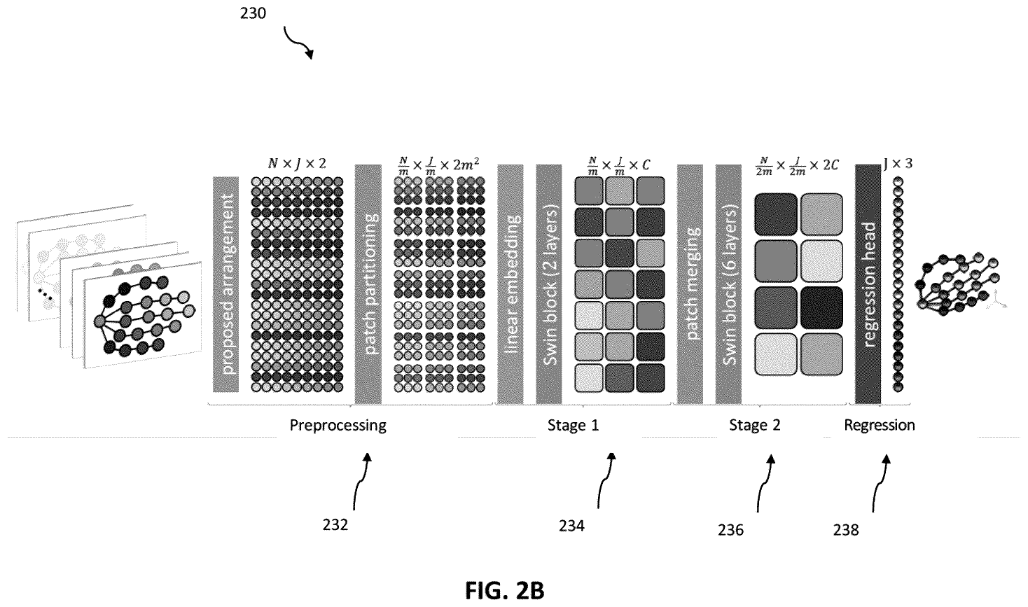

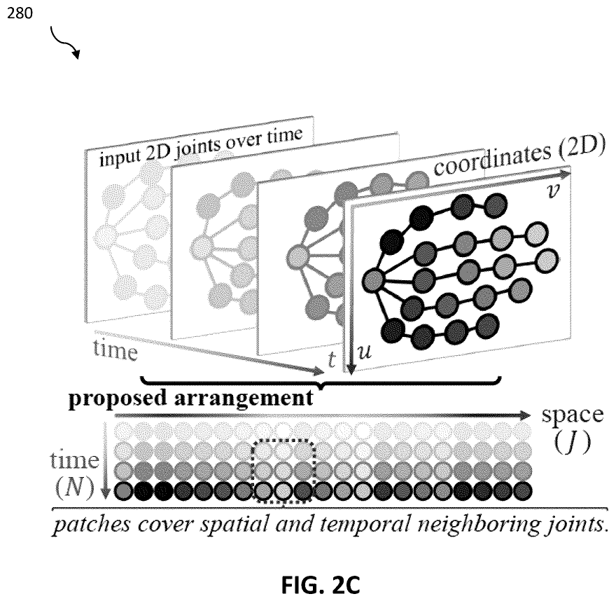

In examples, hand alignment unit 156 may be configured to crop a hand region, for example, based on the bounding box 110 described here. Hand alignment unit 156 may be further configured to perform hand alignment based on the predicted bounding box (e.g., 110) and a rotation angle relative to a pre-defined orientation or direction (e.g., an upward direction). System 150 may further include a hand 2D landmark detection unit 160 (e.g., a software component) configured to generate a plurality of 2D landmarks 162. In some examples, a 2D landmark associated with a hand may include a joint or fingertip (e.g., 202 in FIG. 2A), and the plurality of 2D landmarks may jointly indicate the shape and/or pose of the hand in 2D. In some examples, the plurality of 2D landmarks may be represented in a heatmap. System 150 may further include a 3D pose estimation neural network 164 configured to predict a 3D pose of the hand based on the representation (e.g., heatmap) of the plurality of 2D landmarks and the cropped and/or reoriented hand image. System 150 may further include a gesture classifier 170 configured to determine which class the hand gesture may belong to (e.g., “okay,” “non-okay,” “pinch,” “expand”, “rotate,” etc.) based on the predicted 3D pose of the hand.

With further reference to FIG. 1B, hand detection unit 154 may include a machine learning (MHL) model that may be pre-trained for identifying the area of the image that may include one or more hands and for determining an orientation of the hand, for example, relative to a pre-defined direction (e.g., an upright direction). The MVL model may be implemented using an artificial neural network (ANN) comprising a first plurality of layers trained for extracting features from image 102 and determining, based on the extracted features, which area (e.g., pixels) of the image may include the hand. The ANN may, for example, be trained to generate a bounding shape 110 (e.g., a bounding box) around the image area that may correspond to the hand based on the features extracted from image 102. In some examples, the ANN may further include a second plurality of layers trained for predicting an orientation of the hand based on the features extracted from image (e.g., 102). The ANN may predict the orientation of the hand, for example, an angle (e.g., a number of degrees) between an area of the hand (e.g., an imaginary line drawn through the palm of the hand) and a pre-defined direction (e.g., which may be represented by a vector), and may indicate the predicted orientation in terms of the angle and as an output of the ANN. For example, by processing image 102 through the first and second pluralities of layers, the ANN may output a vector [x1, x2, y1, y2, ori., conf.], where [x1, x2, y1, y2] may define a bounding box surrounding the image area that contains the hand, [ori.] may represent the orientation of the hand, and [conf.] may represent a confidence level or score for the prediction (e.g., prediction of the bounding box and/or the hand orientation). As will be described in greater detail below, information obtained via the orientation-aware hand detection unit 154 may be used to reduce the uncertainty and/or complexity associated with hand shape/pose determination, thereby simplifying the gesture prediction task for the gesture recognition apparatus. This reduces the degrees of freedom of the target data/distribution and helps with recognition accuracy.

As shown in FIG. 1B, hand alignment unit 156 (e.g., a software component) may be configured to at least align the hand as depicted by the image area with the pre-defined direction described above by adjusting the image area (e.g., 110). For example, adjusting the image area may include cropping the image area that contains the hand (e.g., based on bounding shape 110) from image 102 and rotating the cropped image section by the angle determined by hand detection unit 154. In examples, hand alignment may further include scaling (e.g., enlarge/reduce) the cropped image section to a pre-determined size (e.g., 256×256 pixels), e.g., to zoom in or out on the hand. The pre-defined direction used to guide the rotation of the cropped image section may be adjustable, for example, based on how an ML model for predicting a hand gesture based on the cropped image section may have been trained.

Similarly, the image size to which the cropped image section is scaled may also be adjustable based on how the ML model for predicting the hand gesture may have been trained. Through one or more of the rotation or scaling, system 150 may obtain an adjusted image 158 of the hand with a desired (e.g., fixed) orientation and/or size to eliminate the potential ambiguities and/or complexities that may arise from having a variable image orientation and/or image size. Further, by including the image alignment and/or scaling operation of at hand alignment unit 156 as a part of system 150, an ML gesture prediction model trained based on images of a specific orientation and/or size may still be employed to process images of other orientations and/or sizes, for example, by adjusting those images in accordance with the operations described with respect to hand alignment unit 156.

As shown in FIG. 1B, hand 2D landmark detection unit 160 may be configured to detect a plurality of 2D landmarks associated with the hand using the adjusted image 158. In some examples, the plurality of landmarks detected by the hand 2D landmark detection 160 may be pre-defined, for example, to include all or a subset of the joints in a human hand and/or other shape or pose-defining anatomical components (e.g., fingertips) of the human hand. Similar to hand detection unit 154, hand 2D landmark detection unit 160 may also use an ML model. For example, the ML model may also be implemented using an ANN that may be trained for extracting features from the adjusted hand image 158 and determining the respective positions of the landmarks based on the extracted features. The training of such an ANN (e.g., to learn ML model) may be conducted using a training dataset, which may include hand images of the same orientation and/or size, or hand images of different orientations and/or sizes. In either scenario, the orientations and/or sizes of the training images may be adjusted to a pre-defined orientation and/or size that may correspond to the pre-defined direction used to rotate the bounding shape (e.g., 110) and the pre-defined image size used to scale the bounding shape 110, respectively. Through such training and given the adjusted hand image 158, the ANN may generate a landmark heatmap 162. Landmark heatmap 162 may indicate (e.g., with respective probability or confidence scores) the areas or pixels of the adjusted hand image that may correspond to the detected 2D landmarks.

In certain medical environments, such as, e.g., a scan room, the accuracy of hand gesture recognition may be challenging due to difficulty associated with detecting relatively smaller objects (e.g., such as a hand) in images that also depict medical devices and multiple people (e.g., as illustrated by image 102 of FIG. 1A). Further, low-light conditions in scan rooms and/or low resolution in obtained images may negatively affect the accuracy of hand gesture recognition. Accordingly, data augmentations may be performed for hand landmark detection to address the aforementioned issues and/or other issues. In some examples, in training the ML model, the system may simulate low resolution and adjust brightness and contrast to increase the diversity of the training set.

In a non-limiting example, for each training image Iori, a low-resolution version of the image may be generated as:

$${\hat{I}}_{lr}^{-} = {K}_{↑}^{s} ( min ( {K}_{↓}^{s} ( 1 , b \cdot {I}_{ori} ) ) + n )$$

where K_{⊆}^{s} and K_{↑}^{s} represent down-sampling and up-sampling operations of scale s∈{1, 2, 4, 8}, n is Gaussian noise, and b∈(0.75, 1.25) is a ratio randomly sampled to adjust the image brightness. Accordingly, the ML model for hand 2D landmark detection may be trained to infer the heatmaps of 2D landmarks (e.g., 162) using the aligned hand images (e.g., 158). In some examples, landmark heatmaps may use a Gaussian-like kernel to represent a landmark, where the coordinates of a landmark on the image can be extracted from the highest value of the corresponding heatmap kernel. In some examples, the ML model in hand 2D landmark detection unit 160 may include a regression-based model, where each channel of the heatmap corresponds to a landmark.

In FIG. 1B, 3D pose estimation network 164 may be configured to estimate a 3D pose of the hand (e.g., in which one or more fingers and joints of the hand are positioned and/or oriented to form a gesture). The 3D pose of the hand may be indicated by a plurality of 3D landmarks of the hand. The 3D pose estimation may address the ambiguity of hand gestures from a 2D space and improve the accuracy and robustness of gesture recognition, for example, allowing it to be used for locating a scanning region and triggering the movement of a medical device to a desired position. As shown in FIG. 1B, 3D pose estimation network 164 may use a dual-modality 3D pose estimation model, which takes two modalities, the hand region image (e.g., 158) and 2D hand landmarks (e.g., landmark heatmap 162), as inputs, and output latent features and/or 3D land landmarks for hand gesture classification. The latent features may be used by gesture classifier 170 to determine a gesture of the person, while training 3D pose estimation network 164 to generate the 3D landmarks together with the latent features may make the latent features more meaningful and improve the accuracy of the gesture classification. The 3D landmarks may also be used for visualization and/or interaction with 3D shapes in a downstream application.

In FIG. 1B, gesture classifier 170 may be configured to estimate the hand gesture based on the latent features extracted by 3D pose estimation network 164 from adjusted hand image 158 and/or heatmap 162. The latent features may include 3D hand features, for example. Additionally, or alternatively, 3D pose estimation network 164 may output a global camera translation associated with the camera used to capture the input image 102. The global camera translation may include a matrix that can be used to transform points in camera coordinates to world coordinates (or vice versa). A combination of the global camera translation and the 3D landmarks described herein may bring those landmarks into a real world coordinate system, such that the landmarks may be used to interact with one or more 3D shapes in a downstream application.

FIG. 1C illustrates an example of a 3D hand pose estimation model 180, which may be implemented in hand 3D pose estimation network 164 or gesture classifier 170. As shown in FIG. 1C, 3D hand pose estimation model 180 may include multiple sections, such as head section 182, middle section 184, and tail section 186. 3D hand pose estimation model 180 may also include a fusion section 188. The head section 182 may be duplicated to multiple sub-sections (e.g., 182-1, 281-2) to respectively receive multiple inputs, such as the hand image (158 in FIG. 1B) and landmark heatmap (162 in FIG. 1). The multiple sub-sections (e.g., 182-1, 182-2) may respectively output a feature map associated with the hand image and a feature map associated with the landmark heatmap. The feature map associated with the hand image may be expressed as X_{rgb} ∈R^{C×H×W} and the feature map associated with the landmark heatmap may be expressed as Xₗₘₖ∈R^{C×H×W}.

In FIG. 1C, the fusion section 188 may be configured to fuse the feature map associated with the hand image and the feature map associated with the landmark heatmap, which may be concatenated as X_{rgb|lmk}∈R^{2C×H×QW}. In fusion section 188, the concatenated feature map may be provided to multiple convolution blocks (e.g., two blocks) to output the value V_{rgb|lkm} and weight W_{rgb|lmk}. The fusion section 188 may further include a self-attention model (e.g., a Hadamard model) to determine the product of the value and weight \hat{X}_{rgb|link}−V_{rgb51 lmk}⊙W_{rgb|lmk}. Thus, the output of the fusion section 188 may be expressed as:

$${\hat{X}}_{att} = {f}_{{θ}_{v}} ( {X}_{cat} ) ⊙ {f}_{{θ}_{w}} ( {X}_{rgb \vert lmk} ) , \\ with {X}_{rgb \vert lmk} = {X}_{rgb} ‖ {X}_{lmk} ,$$

where f may represent convolution blocks with parameters θv and θw, respectively, X_{cat} may represent concatenated features, ⊙ may represent a Hadamard product, and II may represent a concatenation operation.

As shown in FIG. 1C, the product of the values and weights described herein may be passed through the middle section 184. Tail section 186 may be duplicated to multiple sub-sections (e.g., 186-1 and 186-2) to output two sets of latent features of the hand, respectively. The latent features may include a set of visual features (e.g., shape and/or pose of a hand) extracted from a hand image and landmark heatmap, for example. As shown in FIG. 1C, the latent features may be expressed as a first set of features Fₗ∈R¹²⁸⁰, which may be used for determining the local 3D landmarks and gesture, and a second set of features F₆∈R²⁸⁰, which may be used for determining the global camera translation. 3D hand pose estimation model 180 may include additional sub-neural networks 190 (e.g., 190-1, 190-2, and 190-3) to respectively predict the 3D landmarks, gesture class, and global camera translation.

In examples, each of the sub-neural networks (190-1, 190-2, 190-3) may be a two-layer MLP (multilayer perceptron), which may include multiple fully connected layers. In non-limiting examples, the number of joints indicated by the 3D landmarks may be 21, although it is appreciated that other suitable numbers may also be possible. Accordingly, the predicted 3D landmarks may have a dimension of 21×3, the gesture class may have a dimension 1×1, and the global camera translation may have a dimension of 1×3. As shown in FIG. 1C, the features Fₗ∈R¹²⁸⁰ are provided to the sub-neural networks 190-1, 190-2 for predicting the 3D landmarks and gesture class, where features F_{g}∈R¹²⁸⁰ are provided to sub-neural network 190-3 to determine the global camera position.

Although sub-neural networks 190-1, 190-2, 190-3 are shown in FIG. 1C as being parts of the ML model shown therein, it should be appreciated that any of these sub-neural networks may be implemented independently. For example, sub-neural network 190-2 may be implemented in a separate ML model (e.g., gesture classifier 170 in FIG. 1B) configured to use the latent features determined by 3D post estimation network 164 (e.g., 3D hand pose model 180) and predict the gesture class (e.g., among a plurality of pre-defined gestures) to which the hand gesture belongs. Returning to FIG. 1B, gesture classifier 170 may be implemented in various configurations, such as by sub-neural network 190-2 (FIG. 1C). Additionally, and/or alternatively, gesture classification may use any suitable machine learning model, e.g., ANN as described above and further herein. Additionally, and/or alternatively, gesture classification may use any traditional classifiers to classify the 3D hand features into one of the pre-defined gesture classes.

In example implementations, the ANN described herein may include a transformer with a self-attention mechanism or a convolutional neural network (CNN) that may comprise an input layer, one or more convolutional layers, one or more pooling layers, and/or one or more fully connected layers. For example, the input layer may be configured to receive an input image (e.g., image 102 of FIG. 1A or hand image 158 of FIG. 1B) or any other input (e.g., latent features), while each of the convolutional layers may include a plurality of convolution kernels or filters with respective weights for extracting features from the input image. The convolutional layers may be followed by batch normalization and/or linear or non-linear activation (e.g., such as a rectified linear unit (ReLU) activation function), and the features extracted from the convolution operations may be down sampled through one or more pooling layers to obtain a representation of the features, for example, in the form of a feature vector or feature map.

In example implementations, the CNN may further include one or more un-pooling layers and one or more transposed convolutional layers. Through the un-pooling layers, the features extracted through the convolution operations described above may be up-sampled, and the up-sampled features may be further processed through the one or more transposed convolutional layers (e.g., via a plurality of deconvolution operations) to derive an up-scaled or dense feature map or feature vector, which may then be used to generate a heatmap or a mask indicating a plurality of landmarks associated with a hand and/or an orientation of the hand. The ANN may be implemented in a similar manner for other types of input, such as latent features.

In some examples, the system as described in various embodiments of the present disclosure may be configured to track the movement of a hand or any part thereof (e.g., fingers, joints, palm center etc.) in 3D to recognize the gesture indicated by the hand. In non-limiting examples, the system may manipulate medical images based on the hand gesture. For example, a person in medical environment may give a “pinch” gesture by closing their thumb and index finger together, and the system described herein may recognize the gesture by tracking the movements of the thumb and the index finger in three dimensions.

In non-limiting examples, the system may zoom in and/or out on a medical image based on a recognized hand gesture. For example, a person may give a “pinch” gesture and the system may map the relative distance between the person's thumb and index finger to determine extent to which the medical image may be expanded or shrunk on a display screen (e.g., the thumb and index finger being closer to each other may lead to zooming out on the image, and moving the thumb and index finger apart may lead to zooming in on the image). In non-limiting examples, the system may rotate medical images based on a hand gesture. For example, a person may use a finger to indicate a “number one” gesture and then rotate the finger (e.g., along x, y, and z directions) to indicate a request or command to rotate a medical image correspondingly. The person may also move the finger to the left, right, up, or down to control the rotation of the medical image. Such acts may be tracked by the system based on the angular movement and/or orientation of the person's finger in three dimensions.

In some examples, system 100 (e.g., as shown in FIG. 1A) may include a calibration system for adjusting the sensitivity and response to hand gesture recognition to accommodate variations in hand sizes, dexterity, and/or environmental conditions. In some examples, system 100 may include a display (e.g., 106 in FIG. 1A) to provide visual cues to guide a person in performing specific hand gestures for image navigation and manipulation.

FIGS. 2A-2C illustrate systems and methods for estimating a 3D hand pose based on spatial and temporal relationships of 2D landmarks extracted from a video that may depict the hand in a sequence of images. As will be described in greater detail below, these systems and methods may combine hand detection, landmark detection, and 3D lifting in estimating the 3D hand pose.

FIG. 2A illustrates an example of 3D hand pose estimation based on a video depicting one or more hands in a medical environment in accordance with embodiments of the present disclosure. As shown in FIG. 2A, a 3D pose estimation model 200 may be used to model the spatial and temporal relationships of 2D landmarks extracted from a sequence of images in the video, and predict the 3D pose of a hand based on the relationships. For example, the 3D pose estimation model 200 may be configured to stack 2D landmarks 202 extracted from the video (e.g., which includes a current image and one or more additional images) and provide the stacked 2D landmark to a machine learned 3D lifting model 204 (e.g., implemented via a vision transformer, which may be a component of the 3D pose estimation model 200) that may be trained to predict the 3D pose of the hand based on the stacked 2D landmarks. The detection of 2D landmarks 202 in the current image and other images of the video may be performed in similar manners as described above with reference to FIG. 1B (e.g., hand 2D landmark detection unit 160), and the stacking of the 2D landmarks across the sequence of images of the video may be performed within a window in which the 3D hand pose is to be predicted.

FIG. 2B illustrates an example machine learning model 230 that may be configured to estimate a 3D hand pose based on a video that depicts the hand in a sequence of images. As shown in FIG. 2B, ML model 230 may be configured to perform the 3D hand pose estimation task in one or more preprocessing stages (e.g., 232), one or more stages (e.g., 234 and 236) associated with deep feature extraction, and one or more regression stages (e.g., 238). During the preprocessing stage 232, extracted 2D hand landmarks may be arranged sequentially across multiple images in the video. For example, hand keypoints (e.g., 2D hand landmarks) associated with consecutive time spots (e.g., neighboring image frames in the video) may share strong intra or inter-relations along the temporal dimension (e.g., past/future positions of a keypoint can inform its current state) and the spatial dimension (e.g., positions of joints in a single frame may be interrelated). As such, during the preprocessing stage 232, input temporal 2D hand landmarks may be arranged into an image-like matrix X∈R_{N×J×2}, where N may denote the number of consecutive frames, J may represent the number of hand joints, and 2 may correspond to normalized uv image coordinates. An example technique for stacking 2D landmarks across multiple image frames is illustrated in FIG. 2C. Using this example technique, temporal and kinematic dimensions may be treated equally such that the interrelationship of neighboring joints in both the temporal and spatial domains may be gathered via a self-attention mechanism (e.g., as part of a transformer) at a later stage.

As shown in FIG. 2B, preprocessing stage 232 may include a patch partitioning layer configured to divide the input data into non-overlapping m×m patches along N and J dimensions. This may result in the tokenization of each patch into a raw-valued feature vector of a specific size (e.g., 3×3×2=18). During stage 234 of FIG. 2B, multiple neural network layers may be used to extract deep features from the stacked 2D landmarks. The multiple neural network layers may include, for example, a linear embedding layer configured to receive the tokenized patches and project them to a suitable dimension denoted as C, and multiple transformer layers configured to perform feature transformation. During stage 238, a patch merging layer may be used to concatenate the features of each group of neighboring patches (e.g., 2×2 in size) and project channel sizes from 4C to 2C. In some examples, zero padding may be applied if the height or width of a patch is an odd value (e.g., if the height or width of the patch cannot be divided evenly by the window size). Stage 236 shown in FIG. 2B may invoke another multilayer transformer that may be coupled to the patch merging layer to further down sample the size of the input based on the values of N and J.

During regression stage 238 shown in FIG. 2B, the patch tokens resulting from the previous operations may be pooled along the N and J dimensions and passed to a linear embedding layer that may estimate the final 3D hand pose \hat{ }y ∈R^{J×3}, where 3 may represent the xyz hand joint coordinates. A loss function based on MPJPE (mean per joint position error) may be used to train ML model 230. Such a loss function may be expressed as follows:

$$ℒ = \frac{1}{J} {\sum }_{j = 1}^{J} {‖ {y}_{j} - {\hat{y}}_{k} ‖}_{2} ,$$

where yⱼ and y\hat{ }ⱼ may represent the ground truth and estimated xyz locations of the j-th hand joint, respectively.

In examples, the resolution of the stacked 2D landmarks described herein may be relatively low (e.g., N×J=21×21), compared to the input image data (e.g., 224×224). Thus, the window size for patch partitioning may be made small (e.g., m=3). The window may be shifted by 1 at each layer. Since the resolution is low, if the number of patches in each dimension cannot be divided evenly by the window size, zero padding may be applied.

In examples, stage 234 of FIG. 2B may include a 2-layer transformer block and stage 236 may include a 6-layer transformer block. The channel size of the hidden layer(s) in stage 234 may be, for example, C=108.

The embodiments described in FIGS. 2A-2C may be advantageous in terms of reduced complexity. For example, the self-attention in the transformer may be performed within each window to achieve a linear computational complexity, while a traditional transformer may have a quadratic computational complexity with respect to the input size. The embodiments described in FIGS. 2A-2C may be advantageous also because the transformer used therein may be configured to jointly model the spatial and temporal interrelationships of the 2D hand landmarks. The self-attention and windowing implemented via the transformer may be highly effective for image processing tasks. Further, the sequential 2D hand landmark arrangement (e.g., 280 shown in FIG. 2C) used for the stacking, may preserve the strong spatial-temporal relations of neighboring joints, which may be leveraged for video-based 2D-to-3D hand pose lifting.

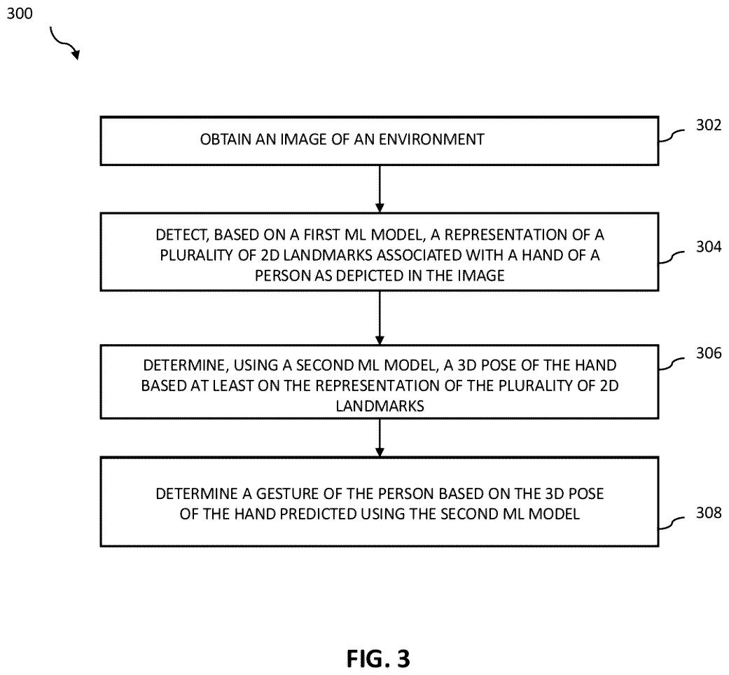

FIG. 3 illustrates an example process 300 that may be implemented by a gesture recognition apparatus for automatically determining a hand gesture. As shown, process 300 may include obtaining an image of an environment (e.g., a medical environment) at 302, and detecting, based on a first machine learning (ML) model, a plurality of 2D landmarks associated with a hand of a person depicted in the image at 304. As described herein, the person may be the operator of a medical device (e.g., an imaging scanner) or a patient, and the operations at 304 may be implemented by hand detection unit 154 and 2D landmark detection unit 160 shown in FIG. 1B. Also as described herein, the plurality of 2D landmarks may be represented in a landmark heatmap.

Process 300 may further include predicting, using a second ML model, a 3D pose of the hand of the person at 306 based at least on the representation of the plurality of 2D landmarks. Process 300 may further include determining, at 308, a gesture of the person based on the predicted 3D pose of the hand. In some embodiments, the operations at 306 and 308 may be implemented respectively in 3D pose estimation network 164 and gesture classifier 170 shown in FIG. 1B. In some embodiments, the operations at 306 and 308 may be implemented using an ML model such as ML model 180 shown in FIG. 1C.

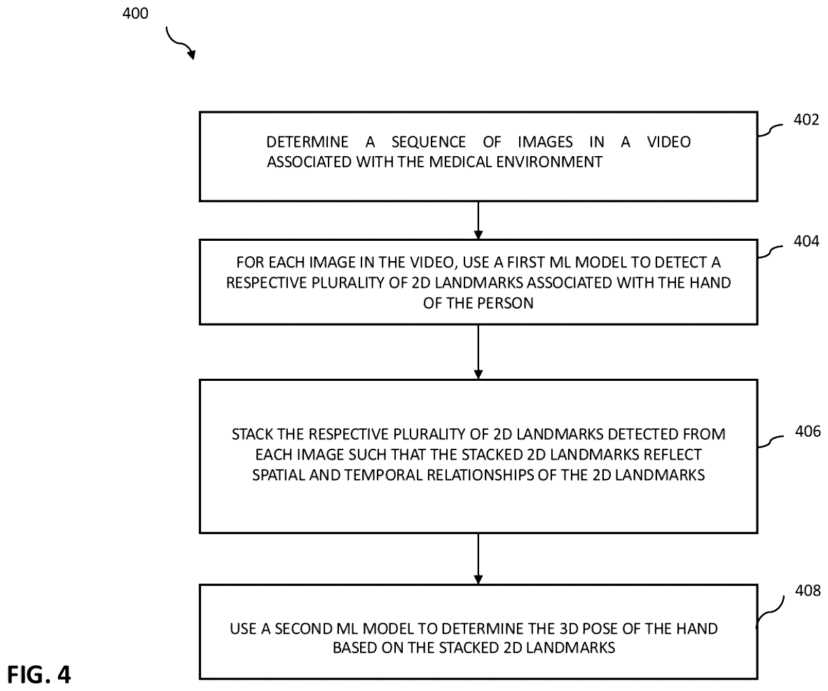

FIG. 4 is a flow diagram illustrating an example procedure 400 for determining a 3D pose of a hand based on a video that depicts the hand in a sequence of images. As shown, procedure 400 may include obtaining the video at 402, wherein the video may include a current image and one or more other images. Procedure 400 may further include detecting, for each of the aforementioned images, a respective plurality of 2D landmarks associated with the hand of the person at 404 using a first ML model. The ML model for detecting the respective plurality of 2D landmarks for each of the images may be the same as the ML model used to detecting the 2D landmarks of the hand at 304 of FIG. 3.

Procedure 400 may further include stacking the respective plurality of 2D landmarks detected from each image at 406, such that the stacked 2D landmarks may reflect spatial and temporal relationships of the 2D landmarks from the images. Examples of the stacked 2D landmarks are shown by 202 of FIG. 2A and an example of a sequential 2D hand landmark arrangement used for the stacking is shown by 280 of FIG. 2C. Procedure 400 may further include predicting, using a second ML model, a 3D pose of the hand of the person based on the stacked 2D landmarks at 408. The second ML model may be implemented in a similar manner as ML model 230 of FIG. 2B.

For simplicity of explanation, processes 300 and 400 may be depicted and described herein with a specific order. It should be appreciated, however, that the illustrated operations may be performed in various orders, concurrently, and/or with other operations not presented or described herein. Furthermore, it should be noted that not all operations that may be included in processes 300 and/or 400 are depicted and described herein, and not all illustrated operations are required to be performed.

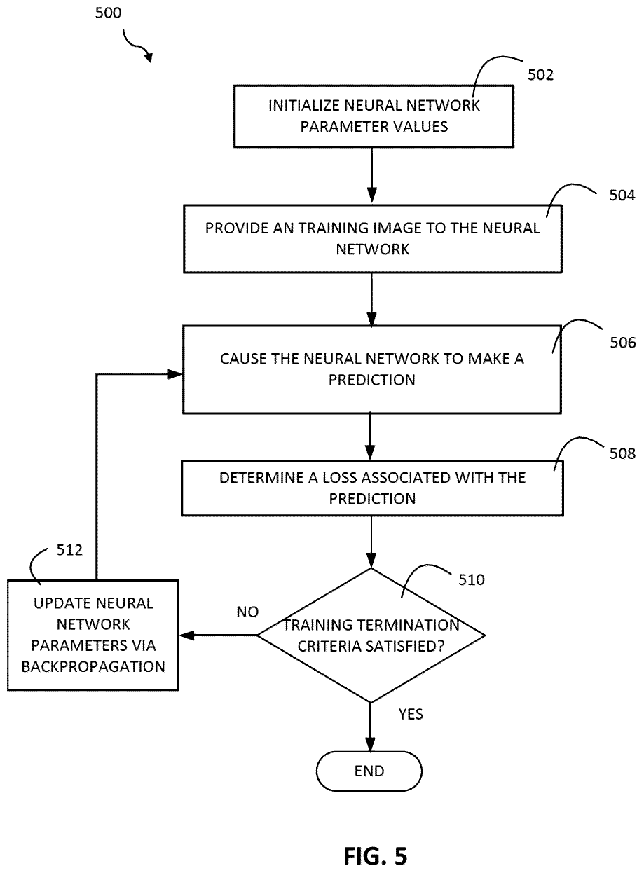

FIG. 5 illustrates operations in an example process 500 that may be associated with training a neural network (e.g., any of the machine learning models described herein) to perform one or more of the tasks described herein. As shown, the training operations may include initializing the parameters of the neural network (e.g., weights associated with the various filters or kernels of the neural network) at 502. The parameters may be initialized, for example, based on samples collected from one or more probability distributions or parameter values of another neural network having a similar architecture. The training operations may further include providing training data (e.g., a training image) to the neural network at 504, and causing the neural network to make a prediction (e.g., 2D landmarks, the 3D pose of a hand, the class of a gesture, etc.) at 506. The prediction made by the neural network may be compared to a ground truth at 508 to determine a loss associated with the prediction. Such a loss may be determined, for example, based on a mean absolute error (MAE), a mean squared error (MSE) or a normalized mean error (NME) between the predicted result and the ground truth. The loss may also be determined based on an L1 norm, an L2 norm, and/or another suitable function.

At 510, the loss determined at 508 may be evaluated to determine whether one or more training termination criteria have been satisfied. For instance, a training termination criterion may be deemed satisfied if the loss(es) described above is below a predetermined threshold, if a change in the loss(es) between two training iterations (e.g., between consecutive training iterations) falls below a predetermined threshold, etc. If the determination at 510 is that the training termination criterion has been satisfied, the training may end. Otherwise, the loss may be backpropagated (e.g., based on a gradient descent associated with the loss) through the neural network at act 512 before the training returns to 506.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

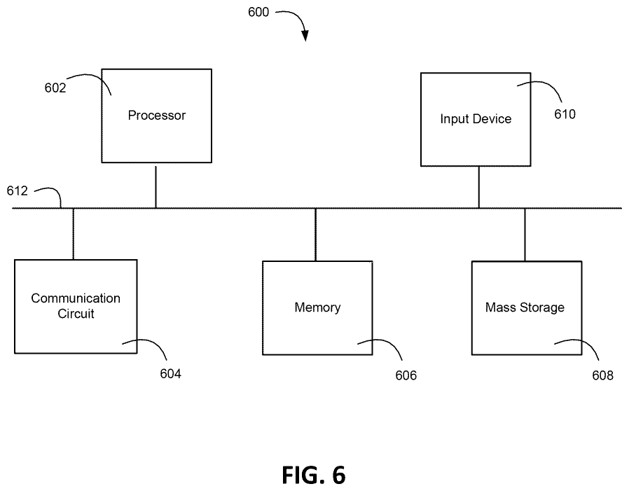

The systems, methods, and/or instrumentalities described herein may be implemented using one or more processors, one or more storage devices, and/or other suitable accessory devices such as display devices, communication devices, input/output devices, etc. FIG. 6 is a block diagram illustrating an example apparatus 600 that may be configured to perform hand gesture recognition tasks described herein. For example, apparatus 600 may include processing apparatus 106 (FIG. 1A). As shown, apparatus 600 may include a processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked, or clustered) with other computation devices, such as shown in FIG. 1A, to perform the functions described herein. And even though only one instance of each component is shown in FIG. 6, a skilled person in the art will understand that apparatus 600 may include multiple instances of one or more of the components shown in the figure.

Various embodiments described above with respect to the accompanying figures provide advantages over existing systems for recognizing hand gestures. For example, the multi-stage pipeline for 3D hand gesture recognition as shown in FIG. 1B includes orientation-aware hand detection (e.g., at 156), illumination-invariant 2D landmark detection (e.g., at 160), and a dual-modality 3D pose and gesture recognition model (e.g., at 164 and 170). These schemes ensure robust and accurate hand gesture recognition and subsequent tasks in a medical environment. In addition, performing 3D hand gesture estimation based on a video (e.g., including the 2D landmark stacking techniques described herein) may further improve the accuracy of 3D pose estimation. The described embodiments herein can be implemented in various applications, including but not limited to virtual and augmented reality, robotics, human-computer interaction, and sign language recognition.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
one or more processors configured to:
obtain an image that depicts at least a hand of a person in a medical environment;
determine, based on a first machine learning (ML) model, a representation of a plurality of two-dimensional (2D) landmarks of the hand as depicted in the image;
predict, based on a second ML model, a three-dimensional (3D) pose of the hand based at least on the representation of the plurality of 2D landmarks of the hand; and
determine a gesture of the person based on the predicted 3D pose of the hand.

2. The apparatus of claim 1, wherein the 3D pose of the hand is predicted further based on the image that depicts the hand, and wherein the second ML model is configured to receive the image that depicts the hand as a first input and the representation of the plurality of 2D landmarks of the hand as a second input.

3. The apparatus of claim 1, wherein the image that depicts the hand of the person is cropped from another image that depicts the hand in the medical environment and re-oriented based on a pre-determined direction.

4. The apparatus of claim 1, wherein the representation of the plurality of 2D landmarks of the hand includes a heatmap that indicates the plurality of 2D landmarks.

5. The apparatus of claim 1, wherein the second ML model comprises:
a first portion configured to determine a first feature map associated with the image that depicts the hand of the person in the medical environment and a second feature map associated with the representation of the plurality of 2D landmarks of the hand;
a second portion configured to fuse the first feature map and the second feature map; and
a third portion configured to predict the 3D pose of the hand of the person based on the fused first feature map and second feature map.

6. The apparatus of claim 5, wherein the second portion of the second ML model comprises a self-attention module.

7. The apparatus of claim 5, wherein the third portion of the second ML model is further configured to determine a global camera translation associated with the image that depicts the hand of the person.

8. The apparatus of claim 1, wherein the one or more processors are further configured to control a medical device or manipulate a medical scan image based on the determined gesture of the person.

9. The apparatus of claim 8, wherein the one or more processors being configured to manipulate the medical scan image comprises the one or more processors being configured to zoom in or out on the medical scan image, or to rotate the medical scan image.

10. The apparatus of claim 1, wherein the image is obtained based on a video associated with the medical environment, wherein the video includes a plurality of additional images that depicts the hand of the person in the medical environment, and wherein the one or more processors are further configured to:
determine, based on the first ML model, respective plurality of 2D landmarks of the hand as depicted by each additional image of the video;
stack the respective plurality of 2D landmarks of the hand associated with each additional image of the video such that the stacked 2D landmarks reflect spatial and temporal relationships of the 2D landmarks in the video; and
determine the 3D pose of the hand further based on the stacked 2D landmarks.

11. The apparatus of claim 10, wherein the second ML model comprises:
a patch partitioning portion configured to divide the stacked 2D landmarks into a plurality of non-overlapping patch areas; and
a transformer coupled to the patch partitioning portion and configured to extract 3D features based on the plurality of non-overlapping patch areas, wherein the 3D pose of the hand is predicted based at least on the 3D features.

12. A method of estimating hand gestures, the method comprising:
obtaining an image that depicts at least a hand of a person in a medical environment;
determining, based on a first machine learning (ML) model, a representation of a plurality of two-dimensional (2D) landmarks of the hand as depicted in the image;
predicting, based on a second ML, model, a three-dimensional (3D) pose of the hand based at least on the representation of the plurality of 2D landmarks of the hand; and
determining a gesture of the person based on the predicted 3D pose of the hand.

13. The method of claim 12, wherein the image that depicts the hand of the person is cropped from another image that depicts the hand in the medical environment and re-oriented based on a pre-determined direction, wherein the 3D pose of the hand is predicted further based on the image that depicts the hand, and wherein the second ML model is configured to receive the image that depicts the hand as a first input and the representation of the plurality of 2D landmarks of the hand as a second input.

14. The method of claim 12, wherein the representation of the plurality of 2D landmarks of the hand includes a heatmap that indicates the plurality of 2D landmarks.

15. The method of claim 12, wherein the second ML model comprises:
a first portion configured to determine a first feature map associated with the image that depicts the hand of the person in the medical environment and a second feature map associated with the representation of the plurality of 2D landmarks of the hand;
a second portion configured to fuse the first feature map and the second feature map; and
a third portion configured to predict the 3D pose of the hand of the person based on the fused first feature map and second feature map.

16. The method of claim 15, wherein the third portion of the second ML model is further configured to determine a global camera translation associated with the image that depicts the hand of the person.

17. The method of claim 12, further comprising controlling a medical device or manipulating a medical scan image based on the determined gesture of the person.

18. The method of claim 17, wherein manipulating the medical scan image comprises zooming in or out on the medical scan image, or rotating the medical scan image.

19. The method of claim 12, wherein the image is obtained based on a video associated with the medical environment, wherein the video includes a plurality of additional images that depicts the hand of the person in the medical environment, and wherein the method further comprises:
determining, based on the first ML model, respective plurality of 2D landmarks of the hand as depicted by each of the additional images of the video;
stacking the respective plurality of 2D landmarks of the hand associated with the each of the additional images of the video such that the stacked 2D landmarks reflect spatial and temporal relationships of the 2D landmarks in the video; and
determining the 3D pose of the hand further based on the stacked 2D landmarks.

20. The method of claim 19, wherein the second ML model comprises:
a patch partitioning portion configured to divide the stacked 2D landmarks into a plurality of non-overlapping patch areas; and
a transformer coupled to the patch partitioning portion and configured to extract 3D features based on the plurality of non-overlapping patch areas, wherein the 3D pose of the hand is predicted based at least on the 3D features.
